(19) United States
(12) Patent Application Publication
(10) Pub. No.: US 2016/0349235 A1
(43) Pub. Date: Dec. 1, 2016

(54) System and Method for Analysis of Protein Migration in Serum Protein Electrophoresis

(71) Applicant: Joshua A. Bornhorst, Little Rock, AR (US)

(72) Inventor: Joshua A. Bornhorst, Little Rock, AR (US)

(21) Appl. No.: 15/165,868

(22) Filed: May 26, 2016

Related U.S. Application Data
(60) Provisional application No. 62/166,454, filed on May 26, 2015.

(51) Int. Cl. G01N 33/487; G01N 27/447

(52) U.S. Cl. CPC G01N 33/48707; G01N 27/44791; G01N 27/4473; G01N 2333/47

(57) ABSTRACT

A system and method for analyzing protein migration in serum protein electrophoresis is disclosed. A serum sample from a subject is collected and serum protein electrophoresis is performed to separate the plurality of proteins in the serum sample. A result of the serum protein electrophoresis is generated and displays a series of peaks. The peak distance between the albumin peak and another normal SPEP protein peak (alpha-1, alpha-2, beta-1, beta-2 or gamma) is measured and compared against the peak distance between the albumin peak and another peak of interest. By dividing the peak distances, a relative migration ratio is calculated. This migration ratio can be used to quantitatively describe migration of peaks in serum protein electrophoresis and to normalize variation in gel migrations between different serum protein electrophoresis measurements, which can be utilized to aid in the interpretation of the serum protein electrophoresis.

### CROSS-REFERENCE TO RELATED APPLICATIONS

This application claims the benefit of U.S. Provisional Application No. 62/166,454, entitled “Reproducible Protein Migration Measurement in Serum Protein Electrophoresis” and filed on May 26, 2015. The complete disclosure of said provisional application is hereby incorporated by reference.

### STATEMENT REGARDING FEDERALLY SPONSORED RESEARCH OR DEVELOPMENT

Not applicable

### BACKGROUND OF THE INVENTION

Detection and monitoring of monoclonal gammopathy in multiple myeloma and other related plasma cell disorders routinely includes serum protein electrophoresis (SPEP). SPEP can be undertaken by either gel electrophoresis and/or capillary zone electrophoresis. The observation of monoclonal protein density is typically achieved by visualization of the protein density in gel electrophoresis or ultraviolet absorbance tracing in capillary zone electrophoresis to distinguish proteins of interest among the other normal or administered therapeutic proteins in serum. In serum protein gel electrophoresis, the migration of the protein on a gel can be visualized after gel protein staining. In capillary electrophoresis, the time at which a protein passes an absorbance window is measured and a tracing of the absorbance of ultraviolet light representing the presence of protein in generated. Generally, in both systems the differences in the separation of proteins in the gel or the tracing is commonly referred to as protein migration. Accurate identification of the protein of interest is needed to obtain serum concentration of the protein of interest in SPEP. One drawback to this is that the migration of normal serum proteins and other interfering proteins or therapeutic antibodies can occlude and inhibit accurate measurement of monoclonal protein (M protein) density which is often used to monitor disease progression and rely on consistent identification of the M protein among other proteins present in SPEP.

Distributions of M-protein gel migrations are useful in the consideration of the utility of SPEP M-protein determination versus other methods of myeloma protein densities such as Hevylite determinations (The Binding Site, San Diego Calif.). Concentration determinations of M-protein are generated utilizing SPEP determinations of protein migration and are commonly used to monitor and diagnose diseases of plasma dyscrasia including multiple myeloma and monoclonal gammopathy of unknown significance. The volume of the peak identified as M-protein is divided by the total peak volumes in the SPEP and then multiplied by the total protein concentration of the serum sample. Thus, identification of the M-protein peak is critical to determination of M-protein concentrations. The presence of oligoclonal immunoglobulin banding associated with normal immune response or treatment may complicate proper M-protein identification. Clonal escape can also result in potential migration changes. Furthermore, identification of abnormalities in the concentrations of other proteins such as alpha 1-antitrypsin can be also indicative of pathological disorders. Accurate identification of the migration of proteins normally in the serum or therapeutic proteins (such as administered monoclonal antibodies) can assist in diagnosis, monitoring and evaluation of therapy. Finally the presence of interfering proteins such as fibrinogen can also be potentially identified by quantitative description of characteristic migration.

Currently, gel migration patterns are visually evaluated and may be difficult to determine exact migrations of proteins across serum protein electrophoresis on a patient over time in different SPEPs. At present, there are no reliable methods described for the quantitative, reproducible description of protein migrations in SPEP analysis (or inter-SPEP analysis), including M-protein migration. Absolute protein migrations vary from SPEP to SPEP, due to differences in gel composition, voltage, and run times. Thus, a reproducible method is needed to normalize inter-SPEP protein migrations in an individual over time and is advantageous to potential disorder diagnosis and monitoring.

The limitations of the prior art are overcome by the present invention as described below. Use of the present migration ratio method gives a stable, precise, objective, and consistent method to elucidate expected M-protein migrations which may be integrated into SPEP monitoring protocols for disease progression. The method described here to quantify protein peak migrations is reproducible between different SPEPs run on the same individual and is applicable to both gel electrophoresis SPEP and capillary zone electrophoresis SPEP. This method is applicable methods for SPEP determination including gel electrophoresis and capillary electrophoresis. This method can be used to quantitatively, accurately and reproducibly map the migration of gel proteins and is useful for reproducible interpretation of serum protein electrophoresis in multiple myeloma and other disorders. In principle this method to describe protein migrations could be extended to other sample types that contain sufficient protein such as cerebral spinal fluid or urine.

### BRIEF SUMMARY OF THE INVENTION

The present invention is directed to a system and method for analyzing protein migration in serum protein electrophoresis. A serum sample from a subject is collected and serum protein electrophoresis is performed to separate the plurality of proteins in the serum sample. A graphical representation of the serum protein electrophoresis is generated and displays a series of peaks. The peak distance between the albumin peak and another normal SPEP protein peak (alpha-1, alpha-2, beta-1, beta-2 or gamma) is measured and compared against the peak distance between the albumin peak and another peak of interest. By dividing the peak distances, a relative migration ratio is calculated. This migration ratio can be used to quantitatively describe migration of peaks in serum protein electrophoresis and to normalize variation in gel migrations between different serum protein electrophoresis measurements, which can be utilized to aid in the interpretation of the serum protein electrophoresis.

These and other features, objects and advantages of the present invention will become better understood from a consideration of the following detailed description of the preferred embodiments and appended claims in conjunction with drawings as described following:

### BRIEF DESCRIPTION OF THE DRAWINGS

FIG. 1 is a graphical representation illustrating the measurements from SPEP used in the present invention and the striped protein peak is a confirmed M-protein peak in the SPEP tracing shown.

FIG. 2 is a SPEP tracing from a serum sample from a subject taken on Jul. 17, 2007. This subject is different from the subject presented in FIG. 1.

FIG. 3 is a SPEP tracing from a serum sample taken on May 3, 2015 from the same subject as shown in FIG. 2. This subject is different from the subject presented in FIG. 1.

### DETAILED DESCRIPTION OF THE INVENTION

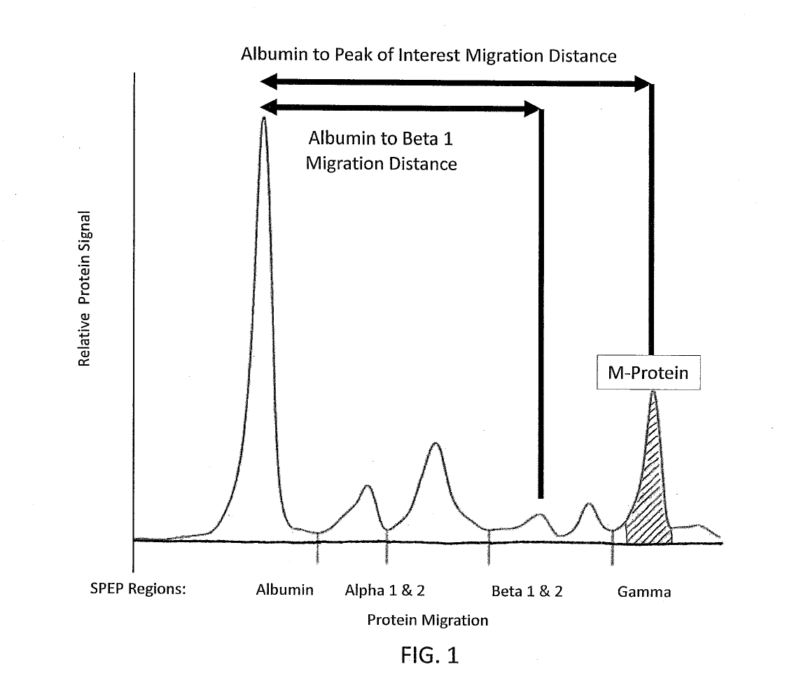

With reference to FIGS. 1-3, the system and method of analyzing protein migration in serum protein electrophoresis of the present invention may be described. As described in more detail below, the present system and method can aid in the reproducible identification and evaluation of the presence of potential monoclonal protein in suspected or confirmed cases of multiple myeloma. Whole blood samples are drawn by phlebotomy from a patient or subject. Following centrifugation of the patient sample to generate serum, a volume of serum is placed in either a gel or a capillary tube. Electrophoresis (ether gel electrophoresis or capillary zone electrophoresis) is performed to separate the proteins. In the case of gel electrophoresis the proteins are stained and densitometry is performed to generate a SPEP result or representation of protein peaks on the gel. Alternatively, in capillary electrophoresis, a SPEP result or representation in the form of a tracing (like shown in FIGS. 1-3) of UV absorbance versus electrophoresis migration time is generated to visualize the proteins present. In both electrophoretic methods, peak migrations in the result can either be measured either by hand for the peak tracings or potentially by aid of a computing device. The SPEP is then examined by a physician and/or other trained personnel for protein abnormalities in the sample. The use of the described system for the determination of relative protein migration ratios could be utilized in to aid the interpretation of the SPEP. For example this would allow for the migration of proteins of interest in previous patient SPEPs to be compared to the current SPEP or to indicate which regions of the subsequent SPEP should be closely examined.

In order to quantitatively and reproducibly describe protein peak gel migrations of monoclonal proteins, a system and method was developed relying on the relative migrations of normal serum proteins compared against observed migration of M-proteins to generate a relative migration ratio. The migration distance (either a physical distance in a SPEP gel or a graphical representation of capillary migration time) can be measured between two peaks. As shown in FIG. 1, the distance between albumin and a normal SPEP protein peak (alpha 1, alpha 2, beta 1, beta 2 or gamma) can be measured and compared against the distance between albumin and another SPEP peak of interest. In principle, the distance between albumin and any normal SPEP peak can be used in the comparison. This is described as a protein migration ratio and can be represented as either (migration of albumin-migration of protein of interest peak distance)/(migration of albumin-migration of normal SPEP peak distance) or as the inverse (migration of albumin-normal SPEP peak distance)/(migration of albumin-migration of protein of interest distance). Either ratio representation is acceptable. This migration ratio system can be used to quantitatively describe migration of peaks in SPEP and also normalizes variation in gel migrations between different SPEP measurements. While the absolute distance between peaks in SPEPs may vary from gel to gel, the relative protein migration ratio should stay constant for SPEP proteins in different gels as alterations in absolute migrations will be normalized by concurrent proportional alteration of the distance between albumin and a normal SPEP peak. These migrations and distances needed to calculate migration ratios could be measured by hand form the SPEP tracings or incorporated in the software utilized to analyze SPEP peaks by a computing device. The calculations to generate ratios can be calculated manually or performed by SPEP electronic analysis.

In order to explore the utility of this method, Sebia (Norcross Ga.) Capillarys capillary SPEP determinations were utilized, although this method is applicable to other SPEP methodologies such as gel electrophoresis which also yield migrations of serum proteins by electrophoresis. A ratio of (albumin-protein peak of interest distance)/(albumin-beta 1 peak distance) was investigated. This ratio was used to map the migrations of peak densities of the alpha-1 and -2, beta-2, and gamma regions in 20 normal individuals, yielding remarkably consistent migration ratios (coefficients of variation of less than 2.3% for beta 2 and gamma inter-individual peak migrations). This was more precise than using the ratio of (albumin-protein peak of interest migration distance)/(albumin-alpha 2 migration distance) for the same set of normal individuals, which resulted in relative inter-individual peak migrations of less than 5% for the beta 2 and gamma regions. Inter-day and Intra-day precision of M-protein migration ratio yielded coefficient of variation of less than 1.5% in an individual with an IgA-Lambda monoclonal protein, and less than 1.4% in an individual with an IgG lambda individual. Finally, an individual gamma with an IgG kappa M-protein exhibited a remarkably stable gel migration over ten SPEPs spanning an 8 year period with a coefficient of variation of less than 1.6%

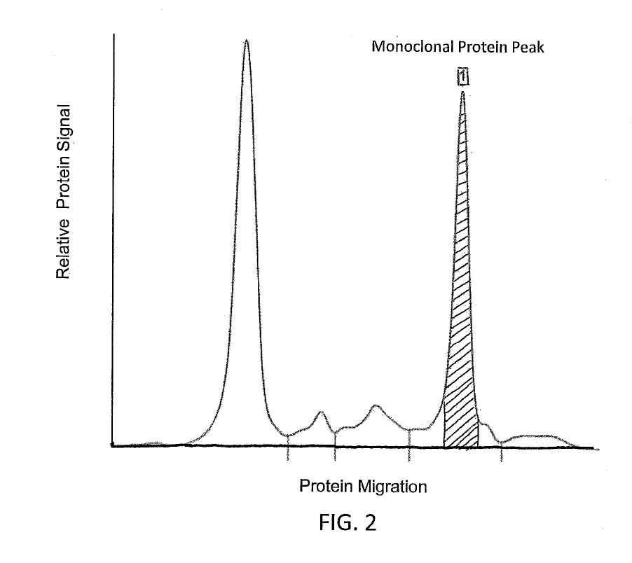

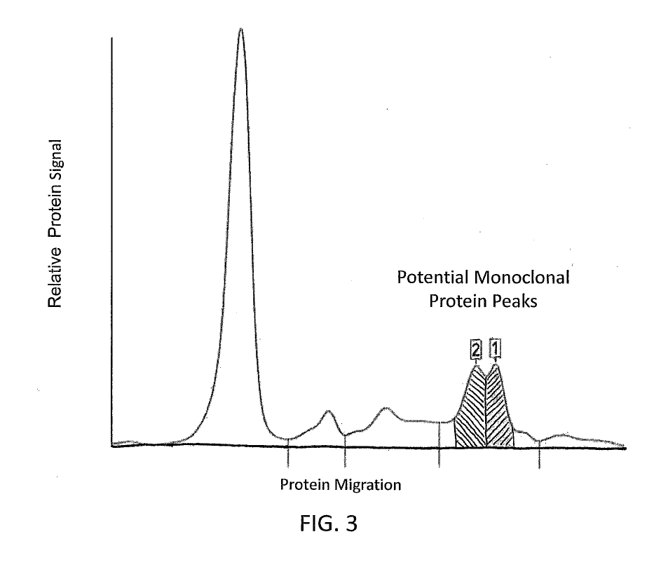

Over 95 individuals with M-proteins were mapped yielding a histogram of relative gel migrations for different monoclonal types. Distributions of M-protein gel migrations are useful in the consideration of the utility of SPEP M-protein determinations. The presence of oligoclonal banding may complicate proper M-protein identification. Clonal escape can also result in potentially migration changes. Use of this migration ratio method gives a stable, precise, objective, and consistent method to elucidate expected M-protein migrations which may be integrated into SPEP monitoring protocols. As is shown in FIGS. 2 and 3, an SPEP tracing pattern was obtained for the same patient a number of years apart. The SPEP of FIG. 2 was used to establish a relative migration ratio of 1.69 (albumin-protein peak of interest distance)/(albumin-beta 1 peak distance) for a monoclonal protein associated with multiple myeloma in the gamma region. In FIG. 3, an SPEP was generated more than 7 years later from the same patient following treatment. Two protein peaks (labeled 1 and 2) are present in the gamma region. As the initial M-protein is of interest in monitoring the presence of disease, it is important to identify the original M-protein migration. This can be difficult to determine by examination of the SPEP. Peak 1 had a relative migration ratio of 1.71 (albumin-protein peak 1 ((as labeled in FIG. 3)) distance)/(albumin-beta-1 peak distance), and peak 2 had a relative migration ratio of 1.57 ((albumin-protein peak-2 (as labeled in FIG. 3) distance))/(albumin-beta-1 peak distance). As it is known that migration ratios vary by less than 2% over time, it strongly indicates that peak 1 is the original monoclonal protein associated with disease, and facilitates pathological interpretation of the SPEP. The striped areas represent the volume of the identified monoclonal peaks used in the subsequent calculation of the concentration of monoclonal protein which is used in disease staging and monitoring.

While this invention may be used with existing SPEP methods and platforms, it represents a novel way of evaluation of SPEP protein migrations (including potential monoclonal proteins), in a reproducible and precise manner. This is useful in evaluation of SPEP electrophorectic patterns and evaluation of monoclonal protein across different SPEPs over time in a patient and helps ensure that the proper peaks are used to assess the amount of monoclonal protein present.

A simpler but potentially less accurate method for the assessment of protein migration is measuring the distance between the albumin peak and the protein of interest. Assuming that the electrophoresis conditions and electrophoresis times were sufficiently similar between SPEP determinations, this can also be used to quantitate protein migration. However variations in electrophoresis voltage, conditions, composition, run time and SPEP representation will limit the accuracy of measuring the difference between albumin and the protein of interest, whereas the protein migration ratio will normalize these potential differences between SPEPs runs. However, if the SPEP conditions are standardized well enough measuring the albumin to protein distance may also be sufficient as well to describe protein migration and may be considered clinically useful.

This method of protein gel migration quantitation can be integrated into existing SPEP methods and techniques, and be used to better interpret and evaluate protein banding patterns, especially in the monitoring and diagnosis of multiple myeloma. The accurate quantitation of protein banks patterns in a single patient over time over multiple gels can be used to better monitor and identify the presence of multiple myeloma. In addition to the evaluation and monitoring of multiple myeloma and other pathological disorders, the present method may be utilized in medical diagnostics for the interpretation of serum protection electrophoresis, automated interpretation of serum protein electrophoretic banding patterns, monitoring of the progression of multiple myeloma, describing migration patterns of monoclonal proteins, evaluation of potential clonal escape of multiple myeloma, and to facilitate the identification of the presence of therapeutic proteins and/or potential interfering proteins.

The present invention has been described with reference to certain preferred and alternative embodiments that are intended to be exemplary only and not limiting to the full scope of the present invention.

We claim:

1. A method for analyzing protein migration in serum protein electrophoresis comprising the steps of:
(a) obtaining a serum sample from a subject, wherein said serum sample comprises a plurality of proteins;
(b) performing serum protein electrophoresis on said serum sample to separate said plurality of proteins;
(c) generating a result of said serum protein electrophoresis comprising a first peak, a second peak, and a third peak, wherein said first peak is associated with presence of albumin in said serum sample and said second peak is associated with the presence of a protein of interest in said serum sample;
(d) determining a first peak distance between said first peak and said second peak;
(e) determining a second peak distance between said first peak and said third peak
(f) calculating a relative migration ratio by dividing said first peak distance by said second peak distance or said second peak distance by said first peak distance.

2. The method of claim 1, wherein said protein of interest is M-protein.

3. The method of claim 1, wherein said third peak is an alpha-1 peak.

4. The method of claim 1, wherein said third peak is an alpha-2 peak.

5. The method of claim 1, wherein said third peak is a beta-1 peak.

6. The method of claim 1, wherein said third peak is a beta-2 peak.

7. The method of claim 1, wherein said third peak is a gamma peak.

8. The method of claim 1, wherein said relative migration ratio is indicative of a disease.

9. The method of claim 1, wherein said result is on a gel.

10. The method of claim 1, wherein said result is a tracing.

11. A system for analyzing protein migration in serum protein electrophoresis comprising:
(a) an electrophoresis apparatus operable to receive a serum sample from a subject comprising a plurality of proteins, wherein said electrophoresis apparatus is operable to perform serum protein electrophoresis on said serum sample to separate said plurality of proteins and to generate a result of said serum protein electrophoresis comprising a first peak, a second peak, and a third peak;
(b) a computing device, wherein said computing device is operable to determine a first peak distance between said first peak and said second peak and to determine a second peak distance between said first peak and said third peak, wherein said computing device is operable to calculate a relative migration ratio by dividing said first peak distance by said second peak distance or said second peak distance by said first peak distance.

12. The system of claim 1, wherein said first peak is associated with presence of albumin in said serum sample and said second peak is associated with the presence of a protein of interest in said serum sample.

13. The system of claim 1, wherein said third peak is an alpha-1 peak.

14. The system of claim 1, wherein said third peak is an alpha-2 peak.

15. The system of claim 1, wherein said third peak is a beta-1 peak.

16. The system of claim 1, wherein said third peak is a beta-2 peak.

17. The system of claim 1, wherein said third peak is a gamma peak.
